(19) United States
(12) Patent Application Publication
(10) Pub. No.: US 2007/0034213 A1
(43) Pub. Date: Feb. 15, 2007

(54) Monitoring and analyzing self-reported pain level in hospital patients

(76) Inventor: David I. Poisner, Folsom, CA (US)

Correspondence Address: BLAKELY SOKOLOFF TAYLOR & ZAFMAN, 12400 WILSHIRE BOULEVARD, SEVENTH FLOOR, LOS ANGELES, CA, 90025-1030, US

(21) Appl. No.: 11/188,265

(22) Filed: Jul. 22, 2005

(51) Int. Cl. A61B 19/00

(52) U.S. Cl. 128/898; 607/46

(57) ABSTRACT

Briefly, a system and method include the use of a patient operated pain recording device and a reception port to analyze current medical condition data, for example an experienced pain level. Current medical conditions can be analyzed with respect to previously stored medical condition data, other patients medical condition data, current actively, and the like.

### BACKGROUND

### Description of the Related Art

The use of information technology in the healthcare field, for example in hospitals, is purported to be approximately a decade behind other business fields. Managing the care of patients, communicating with family and physicians, and connectedness to the network of care providers is often done manually with paper or in person.

For example, to monitor pain levels of a patient, a nurse or other hospital employee frequently asks the patient to rate their pain level on a numerical scale, typically 1-10 or 1-5. The hospital employee may record the level using pen and paper. The hospital employee may also make a decision as to whether pain medication should be administered depending on the pain level and time since the pain medication was last administered. This method requires an employee to directly interact with the patient. Thus, the interaction happens when it is convenient for the employee, rather than at the patient's convenience. This method also may be impractical if the patient is at home. Further, if the employee doesn't keep accurate records or transcribe them to some other format, it may be difficult to share the pain level records with other employees, such as doctors or remote medical consultants.

More use of information technology in the health field is necessary to ensure high quality, low cost patient care.

### BRIEF DESCRIPTION OF THE DRAWINGS

The present invention may be better understood, and its numerous features and advantages made apparent to those skilled in the art by referencing the accompanying drawings.

FIG. 1 illustrates a self-reporting system according to an embodiment of the present invention.

FIG. 2 illustrates a flow diagram for the self-reporting system according to an embodiment of the present invention.

The use of the same reference symbols in different drawings indicates similar or identical items.

### DESCRIPTION OF THE EMBODIMENT(S)

In the following description, numerous specific details are set forth. However, it is understood that embodiments of the invention may be practiced without these specific details. In other instances, well-known methods, structures and techniques have not been shown in detail in order not to obscure an understanding of this description.

References to “one embodiment,” “an embodiment,” “example embodiment,” “various embodiments,” etc., indicate that the embodiment(s) of the invention so described may include a particular feature, structure, or characteristic, but not every embodiment necessarily includes the particular feature, structure, or characteristic. Further, repeated use of the phrase “in one embodiment” does not necessarily refer to the same embodiment, although it may.

As used herein, unless otherwise specified the use of the ordinal adjectives “first,” “second,” “third,” etc., to describe a common object, merely indicate that different instances of like objects are being referred to, and are not intended to imply that the objects so described must be in a given sequence, either temporally, spatially, in ranking, or in any other manner.

Unless specifically stated otherwise, as apparent from the following discussions, it is appreciated that throughout the specification discussions utilizing terms such as “processing,” “computing,” “calculating,” or the like, refer to the action and/or processes of a computer or computing system, or similar electronic computing device, that manipulate and/or transform data represented as physical, such as electronic, quantities into other data similarly represented as physical quantities.

In a similar manner, the term “processor” may refer to any device or portion of a device that processes electronic data from registers and/or memory to transform that electronic data into other electronic data that may be stored in registers and/or memory. A “computing platform” may comprise one or more processors.

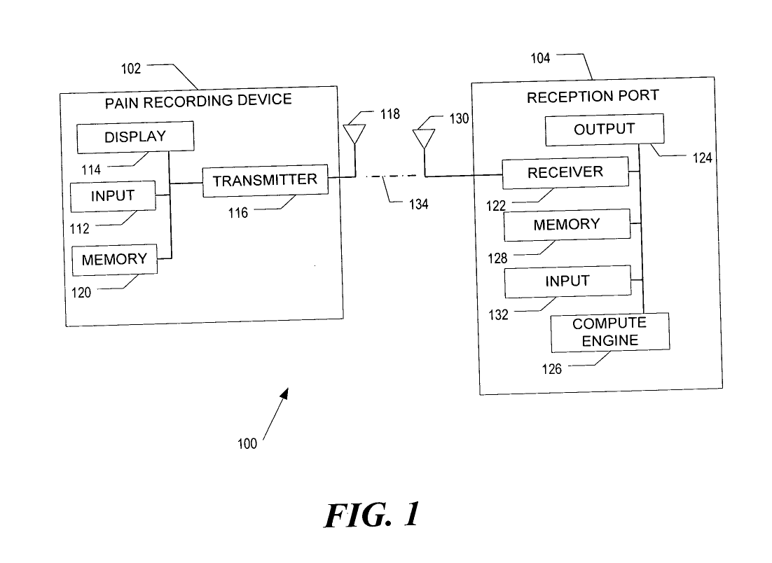

FIG. 1 illustrates a self-reporting system according to an embodiment of the present invention. System 100 includes a pain recording device 102 and a reception port 104. Pain recording device 102 and reception port 104 may communicate with each other wirelessly, via a wire, or via some other communication medium.

Pain recording device 102 may include an input device 112, a display 114, a transmitter 116, an antenna 118, and memory 120. Input device 112 may be a rotating dial, a button, a keypad or the like and is used to enter a current personal medical situation, for example, an experienced pain level. A patient may be provided with a pain recording device 102, for example, a small patient-operated handheld device into which they can enter their current pain level using input device 112. In some embodiments pain recording device 102 has additional buttons that are pressed when a particular event occurs, such as to indicate the patient has woken up, a meal has been completed, or the patient has used the bathroom. Display 114 may be any type of display, for example, a set of LEDs, an LCD, or the like. Display 114 provides visual feedback of the level entered. Transmitter 116 may be configured to transmit the input pain level to reception port 104 via antenna 118, a wired connection, or some other communication medium, for example an optical medium. Memory 120 may be used to store recorded pain levels, for example, when out of communication range or to transmit multiple recorded pain levels at a time.

In one embodiment, pain recording device 102 may be integrated into a computing device provided at each hospital bed. The computing device may include other functions such as television control inputs, a nurse call function, or internet surfing capability. The invention is not limited in this respect. Pain recording device 102 transmits an entered pain level to reception port 104 wirelessly via antennas 118 and 130 or directly via a wire or other communication medium, illustrated as a dotted line, wire 134. In another embodiment, pain recording device 102 may have a direct wired connection to a transmitter that may be connected either via wires or wirelessly to reception port 104. In another embodiment, pain recording device 102 sends values to reception port 104 through the internet.

In another embodiment, pain recording device 102 may be a portable device that a patient carries or wears over a period of time. Pain recording device 102 may have various form factors, for example, a wrist watch type of form factor or a small handheld device. Pain recording device 102 may also include a panic button, for example, if the patient has fallen.

Pain levels may have recurring patterns, such as spikes at specific times during the day or within defined windows after eating, sleeping, using the bathroom, etc. An employee manually recording the current pain level may not recognize these longer-term patterns.

Reception port 104 may include a receiver 122, one or more output devices 124, a compute engine 126, a memory 128, an antenna 130 and an input device 132. Reception port 104 may be connected to other compute engines (not shown) or may be a self contained unit. Output devices 124 may include any type of output device, for example, a video screen, printer, a set of LEDs, a LCD, an internet connection, an audible alarm or the like. Compute engine 126 may be used to analyze the current medical condition and multiple previously recorded conditions. Memory 128 may store analysis software, previously recorded conditions, and the like. Antenna 130 may be used to receive current medical conditions wirelessly from one or more pain recording devices 102.

Reception port 104 may be a remote monitoring site. Reception port 104 may be in the hospital, for example, at a nursing station, or if the patient is at home, the remote monitoring station could be at some other facility. In one embodiment, a local display, such as using LED's mounted on the wall, would indicate one or more recorded pain levels. Thus a hospital employee can quickly see the employee's current pain level. Note that reception port 104 may have multiple output devices 124 that may be in different locations. For example, a bar graph display at a patient's bedside may be used along with a different display at a nurse's station and/or a video screen on a nurse's personal digital assistant (PDA).

Reception port 104 may be a remote monitoring station that can receive messages from multiple pain recording devices 102. Reception port 104 may record the pain levels reported by one or more pain recording devices 102. Software on the remote monitoring station may track the pain levels reported by each pain recording device 102 for the duration of the patient's stay. Software on the remote monitoring station may allow the pain levels for a patient (or several patients) to be displayed in tabular or graphical form. In one embodiment, software on the remote monitoring station provides alerts to hospital employees if a patient's pain level exceeds a defined threshold for some defined period of time, or if the pain level does not decrease by a defined amount after pain medication has been administered. In one embodiment, software on the remote monitoring station allows the hospital employee to enter the time of specific events that might influence pain levels, such as meals, sleep, use of bathroom, or administration of pain medication. A manual override can also be performed, such that the hospital employee can enter alternative pain levels, such as if the patient reports an error in an entry or if the patient appears to be not telling the truth. Reception port 104 may have input device 132 for entering this information.

System 100 may include multiple pain recording devices communicating with one or more reception ports 104. The system is not limited in this respect.

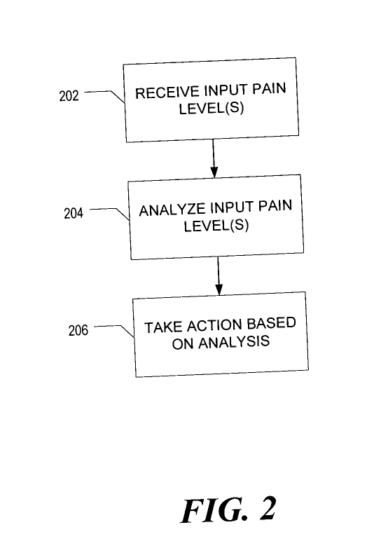

FIG. 2 illustrates a flow diagram for the self-reporting system according to an embodiment of the present invention. A patient's input pain level is received, block 202. The input pain level may include one or more entries, for example, over a period of time, such as a few hours or days. Input pain levels may also include pain levels from multiple patients, for example, for use in a drug effectiveness study. The input pain level may also include other information, for example, current patient activity and the like.

The input is analyzed, block 204. Analysis may include determining a need for increased pain medication dosage levels, or simply another dosage. In one embodiment, software on the remote monitoring station can analyze the pain levels for patterns, such as periodic peaks/valleys, correlation to specific events, such as meals, sleep, or use of bathroom.

Some action is taken based on the analysis, block 206. Action taken based on the result of the analysis may include recommending a pain medication dosage, generation of a written report, generating an alert, or paging a medical professional such as a nurse or a doctor. Software on the remote monitoring station can make recommendations based on the analyzed patterns, such as: medication levels are too low (pain above some threshold) or too high, or fast or slow metabolism of the medication. In one embodiment, software on the remote monitoring station can transmit the patient pain records to another computer. The transmission could be via the internet, other network connection, or other communication media. This allows a doctor at another facility to analyze the pain history for a patient.

According to one embodiment of the present invention, a lack of input received may also be analyzed. For example, if a patient is unconscious or otherwise unable to enter a current pain level, an alert may be issued to a medical professional. Alternatively, the pain recording device may beep or send another similar type of reminder to the patient to enter a pain level.

Automation of the process for recording and analyzing patient pain levels facilitates the sharing of the data among hospital employees that would benefit from the data and analysis.

While the above embodiments have been described in hospital setting, alternate embodiments may be applied to a home setting, where the patient has been sent home from the hospital and still receiving pain medication. The invention is not limited in this respect.

According to one embodiment, pain recording device 102 and reception port 104 are integrated into a single device.

The techniques described above may be embodied in a computer-readable medium for configuring a computing system to execute the method. The computer readable media may include, for example and without limitation, any number of the following: magnetic storage media including disk and tape storage media; optical storage media such as compact disk media (e.g., CD-ROM, CD-R, etc.) and digital video disk storage media; holographic memory; nonvolatile memory storage media including semiconductor-based memory units such as FLASH memory, EEPROM, EPROM, ROM; ferromagnetic digital memories; volatile storage media including registers, buffers or caches, main memory, RAM, etc.; and data transmission media including permanent and intermittent computer networks, point-to-point telecommunication equipment, carrier wave transmission media, the Internet, just to name a few. Other new and various types of computer-readable media may be used to store and/or transmit the software modules discussed herein. Computing systems may be found in many forms including but not limited to mainframes, minicomputers, servers, workstations, personal computers, notepads, personal digital assistants, various wireless devices and embedded systems, just to name a few. A typical computing system includes at least one processing unit, associated memory and a number of input/output (I/O) devices. A computing system processes information according to a program and produces resultant output information via I/O devices.

Realizations in accordance with the present invention have been described in the context of particular embodiments. These embodiments are meant to be illustrative and not limiting. Many variations, modifications, additions, and improvements are possible. Accordingly, plural instances may be provided for components described herein as a single instance. Boundaries between various components, operations and data stores are somewhat arbitrary, and particular operations are illustrated in the context of specific illustrative configurations. Other allocations of functionality are envisioned and may fall within the scope of claims that follow. Finally, structures and functionality presented as discrete components in the various configurations may be implemented as a combined structure or component. These and other variations, modifications, additions, and improvements may fall within the scope of the invention as defined in the claims that follow.

What is claimed is:

1. An apparatus comprising:
an input device to input current medical condition information; and
a transceiver to transmit the current medical condition information to another device.

2. The apparatus as recited in claim 1, wherein the input device comprises a rotating dial.

3. The apparatus as recited in claim 1, wherein the input device comprises one or more buttons.

4. The apparatus as recited in claim 3, wherein the one or more buttons comprise virtual buttons on a touch screen display.

5. The apparatus as recited in claim 1, wherein the transceiver is a wireless transceiver coupled to an antenna.

6. The apparatus as recited in claim 1, wherein the transceiver transmits via a wired connection.

7. The apparatus as recited in claim 1, wherein the transceiver transmits via an optical connection.

8. The apparatus as recited in claim 1, further comprising a display to provide feedback on the current medical condition information.

9. The apparatus as recited in claim 1, wherein the current medical condition information comprises an experienced pain level.

10. The apparatus as recited in claim 1, wherein the input device further to input current activity information.

11. The apparatus as recited in claim 1, further comprising an analyzer to analyze the current medical condition information.

12. The apparatus as recited in claim 11, further comprising a memory to store one or more previous medical condition information, the analyzer further to analyze the current medical condition information in relation to the one or more previous medical condition information.

13. An apparatus comprising:
a receiver to receive current medical condition data;
an analyzer to analyze the current medical condition data; and
an output device to provide output data based on a result of the analyzer.

14. The apparatus as recited in claim 13, further comprising a memory to store one or more previous medical condition data.

15. The apparatus as recited in claim 14, wherein the analyzer further to analyze the current medical condition data in relation to the one or more previous medical condition data.

16. The apparatus as recited in claim 13, the current medical condition data comprises an experienced pain level.

17. The apparatus as recited in claim 16, the current medical condition data further comprising current activity data.

18. The apparatus as recited in claim 13, wherein the output device is a computer screen.

19. The apparatus as recited in claim 13, further comprising an antenna, wherein the receiver is configured to receive wireless transmissions from one or more pain recording devices.

20. A method comprising:
receiving current medical condition data;
analyzing the current medical condition data;
performing an action based on a result of the analyzing.

21. The method as recited in claim 20, wherein the current medical condition data comprises an experienced pain level.

22. The method as recited in claim 20, further comprising analyzing the current medical condition data with previously stored medical condition data.

23. The method as recited in claim 20, wherein the analyzing the current medical condition data comprises comparing the current medical condition data to previously stored medical condition data.

24. The method as recited in claim 20, wherein the analyzing the current medical condition data comprises comparing the current medical condition data with respect to an activity level.

25. The method as recited in claim 20, wherein the analyzing the current medical condition data comprises comparing the current medical condition data to data from other patients.

26. The method as recited in claim 20, wherein the performing the action based on the result of the analyzing comprises changing a medication dosage.

27. The method as recited in claim 20, wherein the performing the action based on the result of the analyzing comprises paging a medical professional.

28. The method as recited in claim 20, wherein the performing the action based on the result of the analyzing comprises generating a report of expected pain levels.

29. An article comprising a storage medium having instructions stored thereon that, when executed by a computing platform, operate to:
receive current medical condition data;
analyze the current medical condition data;
perform an action based on a result of the analyzing.

30. The article as recited in claim 29, wherein the current medical condition data comprises an experienced pain level.

31. The article as recited in claim 29, the instructions, when executed by the computing platform, further operate to analyze the current medical condition data with previously stored medical condition data.

32. The article as recited in claim 29, wherein to analyze the current medical condition data comprises to compare the current medical condition data to previously stored medical condition data.

33. The article as recited in claim 29, wherein to analyze the current medical condition data comprises to compare the current medical condition data with respect to an activity level.

34. The article as recited in claim 29, wherein to analyze the current medical condition data comprises to compare the current medical condition data to data from other patients.

35. The article as recited in claim 29, wherein to perform the action based on the result of the analyzing comprises to change a medication dosage.
